(19) United States
(12) Patent Application Publication
(10) Pub. No.: US 2020/0138676 A1
(43) Pub. Date: May 7, 2020

(54) METHOD FOR PREPARATION OF AN IMPLANT FOR REGENERATING DENTAL TISSUE

(71) Applicants: Gentren, Chungcheongnam-do (KR); Dankook University Cheonan Campus Industry Academic Cooperation Foundation, Chungcheongnam-do (KR)

(72) Inventors: Hae-Won Kim, Chungcheongnam-do (KR); Ho-Jin Moon, Chungcheongnam-do (KR); Jung-Hwan Lee, Chungcheongnam-do (KR)

(21) Appl. No.: 16/533,179

(22) Filed: Aug. 6, 2019

(30) Foreign Application Priority Data
Oct. 12, 2018 (KR) 10-2018-0121744

(51) Int. Cl. A61K 6/06

(52) U.S. Cl. CPC A61K 6/0612; A61K 6/0631

(57) ABSTRACT

The present invention relates to a dental cement composition including a tricalcium silicate powder and a dicalcium silicate powder, a method for preparation of an implant for regenerating a tissue including a tricalcium silicate powder and a dicalcium silicate powder, and a kit for preparing a dental cement including a tricalcium silicate powder and a dicalcium silicate powder.

### CROSS REFERENCE TO RELATED APPLICATIONS

This application claims priority to Korean Patent Application No. 10-2018-0121744, filed on Oct. 12, 2018, the entire content of which is hereby incorporated by reference herein.

### BACKGROUND OF THE INVENTION

### 1. Technical Field

The present invention relates to a dental cement composition comprising a tricalcium silicate powder and a dicalcium silicate powder, a method for preparation of an implant for regenerating a tissue comprising a tricalcium silicate powder and a dicalcium silicate powder, and a kit for preparing a dental cement including a tricalcium silicate powder and a dicalcium silicate powder.

### 2. Description of the Related Art

Mineral trioxide aggregate (MTA), which has been developed based on Portland cement, consists of three main inorganic components of tricalcium silicate (C3S), dicalcium silicate (C2S), and tricalcium aluminate (C3A) and is a promising material for dental regeneration cement. Calcium silicate cement, MTA, has been widely used in dental clinics due to its excellent dental bio-regeneration potential, bioactivity, sealability, and biocompatibility.

Since MTA is generally produced by removing a toxic metal complex from Portland cement, Portland cement and MTA have no difference in cytotoxicity due to their similar compositions except for bismuth oxide (20% to 25%). Since trace elements such as arsenic, chromium, and lead are lower in MTA than in Portland cement, MTA exhibits better proliferation and regeneration ability. However, heavy metal elements such as magnesium, iron, arsenic, chromium, and lead have been found as a cause of toxicity of MTA, and many efforts have been made to eliminate these unnecessary elements.

However, little is known about the exact role of each component in MTA. In particular, a toxic effect of C3A, one of the major components of MTA which is capable of releasing aluminum ions, has not been examined in detail.

As a result of efforts to solve these problems, the inventors of the present invention have contrived an implant having reduced toxicity, which can remarkably reduce hardening time by preparing an implant using a manufacturing method which is different from a conventional method.

### SUMMARY OF THE INVENTION

An object of the present invention is to provide a dental cement composition comprising tricalcium silicate (C3S), dicalcium silicate (C2S), or both in which an individual particle has a mean diameter of 30 μm to 60 μm.

Another object of the present invention is to provide a method for preparation of an implant for regenerating a tissue comprising: a step 1-1 of preparing a tricalcium silicate powder; a step 1-2 of preparing a dicalcium silicate powder; and a step 2 of adding an aqueous solvent to the powder prepared in the step 1-1, the powder prepared in the step 1-2, or a mixture thereof and hardening the powder.

Yet another object of the present invention is to provide a kit for preparing a dental cement including a tricalcium silicate powder, a dicalcium silicate powder, or both.

The inventors of the present invention individually synthesized three components of MTA to solve a toxicity problem of existing MTA, prepared a composition mixed with various components, analyzed properties of an implant prepared therefrom, and then confirmed an effect according to a content of each component. As a result, in particular, the inventers have found that C3A was a major factor in shortening hardening time, but caused significant toxicity in vitro and in vivo. Accordingly, the present invention was contrived to find a composition capable of shortening the hardening time while minimizing the content of C3A and/or a method for preparing such a composition.

Specifically, this will be described as follows. Meanwhile, each description and embodiment disclosed in the present invention can also be applied to each other description and embodiment. That is, all combinations of the various components disclosed in the present invention belong to the scope of the present invention. In addition, the specific description described below may not limit the scope of the present invention.

A first aspect of the present invention provides a dental cement composition comprising tricalcium silicate (C3S), dicalcium silicate (C2S), or both in which an individual particle has a mean diameter of 30 μm to 60 μm.

The dental cement composition of the present invention may further comprise tricalcium aluminate (C3A), and particularly, the tricalcium aluminate may have a mean diameter of 30 μm to 60 μm, and the content of the tricalcium aluminate may be more than 0 wt % to 25 wt % or less based on the total composition weight.

The content of the tricalcium aluminate of the dental cement composition of the present invention may be specifically 45 wt % to 100 wt % and more specifically 45 wt % to 85 wt % based on the total composition weight. The content of the dicalcium aluminate may be specifically 5 wt % to 100 wt % and more specifically 5 wt % to 45 wt % based on the total composition weight.

Based on the total composition weight of the dental cement composition of the present invention, more preferably, the content of the tricalcium silicate may be 45 wt % to 85 wt %, and the content of the dicalcium silicate may be 5 wt % to 45 wt %.

The dental cement composition of the present invention may be used as a biomaterial for dental regeneration.

The term “cement” used in the present invention means a hardening body of a paste which can be used as a bone replacement material, and which is obtained by mixing a powdery solid phase and a liquid phase.

The “hardening or cementation” of the cement means spontaneous hardening of the paste occurring without artificial treatment at room temperature or body temperature, and the paste may be obtained by mixing the solid phase and the liquid phase.

A second aspect of the present invention provides a method for preparation of an implant for regenerating a tissue comprising: a step 1-1 of preparing a tricalcium silicate powder; a step 1-2 of preparing a dicalcium silicate powder; and a step 2 of adding an aqueous solvent to the powder prepared in the step 1-1, the powder prepared in the step 1-2, or a mixture thereof and hardening the powder.

In the method for preparation of the implant for regenerating the tissue of the present invention, based on the total powder, the content of the tricalcium silicate powder may be specifically 45 wt % to 100 wt % and more specifically 45 wt % to 85 wt %, and the content of the dicalcium silicate powder may be specifically 5 wt % to 100 wt % and more specifically 5 wt % to 45 wt %.

The method for preparation of the implant for regenerating the tissue of the present invention further includes a step 1-3 of preparing a tricalcium aluminate powder before the step 2, in which the step 2 may be performed by additionally including the tricalcium aluminate powder in the mixture of the powders of the step 2. Specifically, the content of the tricalcium aluminate powder may be more than 0 wt % to 25 wt % or less based on the total powder.

The tricalcium silicate powder, the dicalcium silicate powder, and the tricalcium aluminate powder may be prepared by a sol-gel process, and in the present invention, the powders are prepared by a sol-gel process, calcination, and heat treatment. Further, an individual particle of the powder may have an average diameter of specifically 30 μm to 60 μm and more specifically 40 μm to 50 μm.

In the method for preparation of the implant for regenerating the tissue of the present invention, a solvent/powder ratio (L/P) used in the step 2 may be specifically 0.2 to 0.4. If the L/P is more than 0.4, mixing is not performed, and if the L/P is less than 0.2, the mixture is too thin and hardening does not proceed.

The method for preparation of the implant for regenerating the tissue of the present invention has an advantage of shortening the hardening time, and the hardening time of the implant may be specifically 1 minute to 50 minutes and more specifically 10 minutes to 40 minutes.

In the method for preparation of the implant for regenerating the tissue of the present invention, the implant may be used for dental regeneration, but is not limited thereto.

The term “implant for regenerating the tissue” used in the present invention is implanted into a tissue defect or a damaged part to fill or supplement the tissue defect or the damaged part, thereby promoting the regeneration of the tissue.

Unlike the conventional manufacturing method, in the manufacturing method of the present invention, the tricalcium silicate powder, the dicalcium silicate powder, and the tricalcium aluminate powder are each prepared and mixed. Accordingly, compared with MTA used as a cement material for regenerating a dental tissue in the related art, it is possible to provide an implant in which the hardening time is significantly shortened while existing physical properties are maintained and toxicity is reduced without an additional toxic material.

A third aspect of the present invention provides a kit for preparing a dental cement including a tricalcium silicate powder, a dicalcium silicate powder, or both.

The kit for preparing the dental cement of the present invention may further comprise a tricalcium aluminate powder, an aqueous solvent, or both. Particularly, the powders may be individually provided or may be provided as a mixture of the tricalcium silicate powder of specifically 45 wt % to 100 wt % and more specifically 45 wt % to 85 wt %, the dicalcium silicate powder of specifically 5 wt % to 100 wt % and more specifically 5 wt % to 45 wt %, and the tricalcium aluminate powder of more than 0 wt % to 25 wt % or less based on the total powder.

The kit may be used by mixing a powder phase with a liquid phase immediately before use to prepare a cement composition in the form of a paste, and then injecting the paste into a desired treatment site. Alternatively, the kit may also be used after preparing a cement in a desired shape by injecting the paste.

A syringe may be used in the treatment.

In addition, the kit of the present invention may further include a biological protein, a drug, or a combination thereof, and examples of the biological protein may include bovine serum albumin, lysozyme, growth factors, and the like, and examples of the drug may include antibiotics and inflammatory agents.

The dental cement composition of the present invention can be hardened within a short period of time and promotes tooth formation differentiation and angiogenesis to induce rapid dental tissue regeneration after the treatment.

The kit of the present invention may be applied to an injection system for repairing and regenerating a dental tissue in tissue engineering, and may be useful for dental treatment in relation to the treatment of dental tissue defects.

The dental cement composition of the present invention is a composition consisting of the same component having a similar content to MTA used as a cement material for regenerating a dental tissue in the related art, but has an effect of significantly shortening hardening time when the implant is prepared due to a difference in a manufacturing method of preparing and mixing three components constituting the composition, that is, a tricalcium silicate powder, a dicalcium silicate powder, and a tricalcium aluminate powder as individual particles. Furthermore, it is possible to provide an implant having reduced toxicity while maintaining physical properties by not containing C3A or minimizing the C3A content.

### BRIEF DESCRIPTION OF THE DRAWINGS

The above and other objects, features, and advantages of the present invention will be more clearly understood from the following detailed description taken in conjunction with the accompanying drawings, in which:

FIG. 1 illustrates results of (a) XRD, (b) SEM, and (c) laser diffraction analysis performed to analyze crystal structures, shapes, and size distributions of MTA, C3S, C2S, and C3A powders;

FIG. 2 is a table illustrating compositions of 14 kinds of cements prepared in the present invention;

FIG. 3 illustrates hardening times of 14 kinds of cements;

FIG. 4 illustrates results of measuring changes in pH of 14 kinds of cements according to incubation time in DW;

FIG. 5 illustrates results of measuring changes in pH of 14 kinds of cements according to incubation time in PBS;

FIG. 6 illustrates results of measuring compressive strength in an SBF solution;

FIG. 7 illustrates results of measuring tensile strength of a diameter in an SBF solution;

FIG. 8 illustrates cell compatibility of 14 kinds of cement extracts with rMSCs;

FIG. 9A is a table illustrating a composition of a cement for an in vivo experiment, FIG. 9B is a schematic diagram illustrating a result of intentionally implanting a rat's front tooth after filling a root-tip with a cement for evaluation of biocompatibility of the cement, and FIG. 9C illustrates an image of μCT after 4 weeks of implantation; and

FIG. 10 illustrates results of H & E staining images after 4 weeks of implantation, and broken-line rectangles indicate magnification regions (C: cement, AB: alveolar bone, T: developmental tooth).

### DESCRIPTION OF THE PREFERRED EMBODIMENTS

Hereinafter, a preferred embodiment is presented in order to assist understanding of the present invention. However, the following embodiment is merely provided to more easily understand the present invention, and contents of the present invention are not limited by the embodiment.

### EXAMPLE 1

### Synthesis of Calcium Powder

C3S, C2S, and C3A were each prepared by a sol-gel process, calcination, and heat treatment according to the related art.

For the preparation of a C3S powder, 0.3 M calcium nitrate tetrahydrate was added to a solution consisting of 70% ethanol, 5% polyethylene glycol (Mw 10000), 1% 1 M hydrochloric acid, and 0.1 M tetraethylorthosilicate (TEOS) and stirred at 60° C. for 3 hours. The mixed solution was kept at 70° C. until gelation occurred and dried at 120° C. for 1 day. The mixed solution was calcined at 500° C. for 1 hour and at 1200° C. for 3 hours, pulverized, and heat-treated at 1450° C. for 8 hours and 10 hours. The obtained powder was pulverized and then sieved using a 45 μm sieve to prepare a C3S powder.

A C2S powder was prepared in the same manner as in the preparation of the C3S powder, except that 0.2 M calcium nitrate tetrahydrate was added, and the mixture was heat-treated at 1000° C.

For the preparation of a C3A powder, 0.2 M aluminum nitrate nonahydrate and 0.3 M calcium nitrate tetrahydrate were added to distilled water (DW) containing 5 wt % polyvinyl alcohol (PVA) and mixed for 5 hours. The mixture was kept at 60 C for 2 days to obtain a gel, and then dried at 120° C. for 24 hours. The gel was pulverized, then heat-treated at 500° C. for 1 hour, pulverized again, and heat-treated at 1350° C. The obtained powder was pulverized and sieved using a 45 μm sieve to prepare a C3A powder. ProRoot MTA and bismuth oxide were specified as controls.

### EXPERIMENTAL EXAMPLE 1

### Analysis of Prepared Powders

The crystal structures of the prepared C3S, C2S, and C3A powders were analyzed by X-ray diffraction (XRD, Ultima IV, Rigaku, Japan). The powders were scanned at a rate of 2° min⁻¹ and a step width of 0.02° in a diffraction range of 2θ=10° to 80° using Cu Kα1 rays at 2 kV and 40 mA.

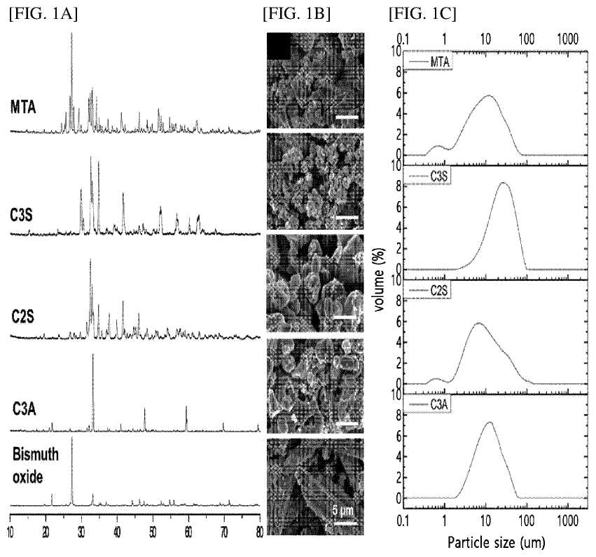

FIG. 1A shows typical XRD peaks of each powder (C3S, C2S, and C3A) and bismuth oxide corresponding to a JCPDS card of each powder, and shows that MTA had a peak of a combination of C3S, C2S, C3A, and bismuth oxide.

The shape of each powder was analyzed by SEM (JEOL-JSM 6510, Tokyo, Japan) at an accelerating voltage of 10 kV. The powder size distribution was also analyzed using a particle size analyzer (Malvern Mastersizer MS2000, Malvern Instruments, Malvern, UK). A suspension was prepared with 50 mL of ethanol and 50 μg of C3S, C2S, and C3A and ProRoot MTA. The D50 (cumulative 50% diameter) of MTA, C3S, C2S, and C3A was determined as a representative size of the powder.

The SEM results showed irregular-shaped powders of several tens of micrometers in all powders (FIG. 1B). Average sizes (D50, cumulative 50% point of diameter) of the powders (MTA, C3S, C2S, and C3A) were determined by laser diffraction to be 9.0 μm, 22.6 μm, 8.4 μm, and 12.0 μm, respectively, and the powders were pulverized and sieved with pores of 45 μm to be compared with one another (FIG. 1C).

### EXAMPLE 2

### Preparation of Dental Cement

The prepared C3S powder, C2S powder, or C3A powder was combined with distilled water (DW), a water-soluble solvent, and then stirred and hardened to prepare a cement. At this time, 10 kinds of cements were prepared by setting a solvent/powder ratio (L/P) to 0.3.

Four kinds of cements were prepared in the same manner except that a single composition of each of C3S, C2S, C3A, and ProRoot MTA was prepared.

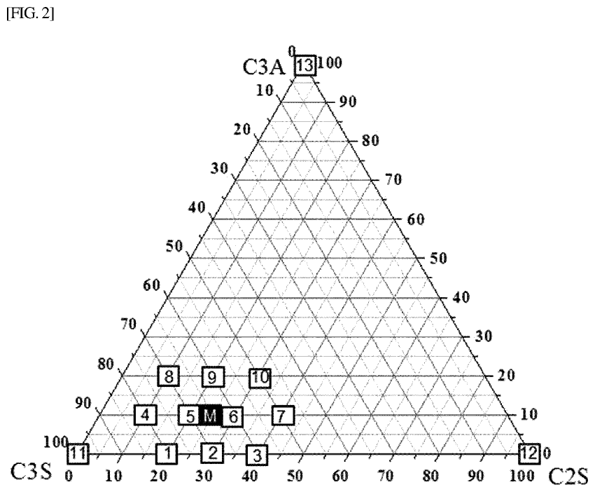

The components for a total of 14 kinds of cements are shown in a ternary graph in FIG. 2.

### EXPERIMENTAL EXAMPLE 2

### Measurement of Physical Properties

Hardening time was measured using a Teflon mold with a diameter of 10 mm and a height of 2 mm in accordance with ISO 6876 (100 g, Ømm Gilmore needle). Within 2 minutes after mixing, the mixture was stored in a constant-temperature water bath at 100% humidity and 37° C. A sample was kept for 5 seconds at a rate of 1.0 mm/min, and initial hardening time was measured with a Gilmore needle having a weight of 100 g and a diameter of 2 mm.

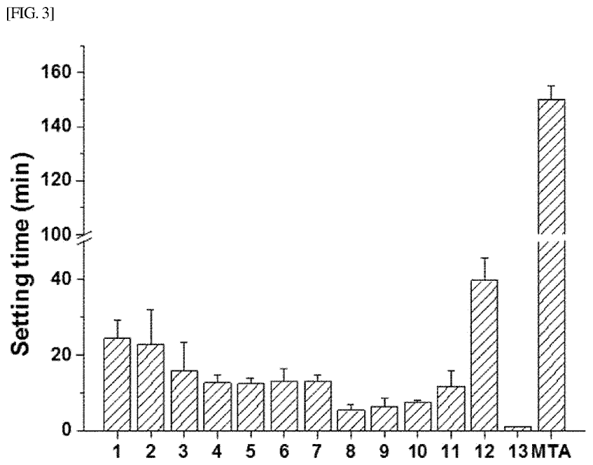

As illustrated in FIG. 3, the initial time was the longest (150±5 minutes) in an MTA cement and the shortest (1±0.2 minutes) in cement 13 consisting of only C3A. It was determined that this is because the addition of C3A quickly hardens C3S and C2S. It could be seen that as an amount of C3A was increased in a cement consisting of a mixture of three powders (0% C3A (1,2,3)>10% C3A (4,5,6,7)>20% C3A (8,9,10)), the hardening time was decreased. This is caused by fast bonding between aluminum ions and a calcium silica powder.

In order to measure a pH change after mixing, samples with an L/P ratio of 0.3 were prepared at a diameter of 6 mm and a height of 2 mm and stored at 37° C. and 100% humidity for 3 hours. Each sample was added to 10 mL of DW and 10 mL of phosphate buffered saline (PBS). The pH was measured at 0, 20, 30, 60, 120, 240, 480, 1440, 2880, and 5760 minutes with a pH meter (Orion 3 star, Thermo Scientific, Singapore).

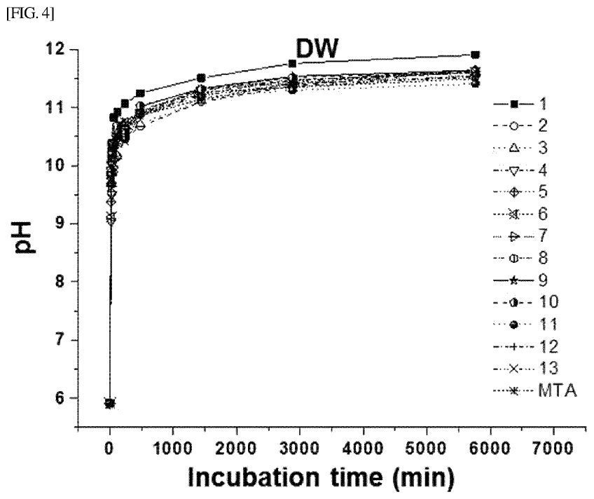

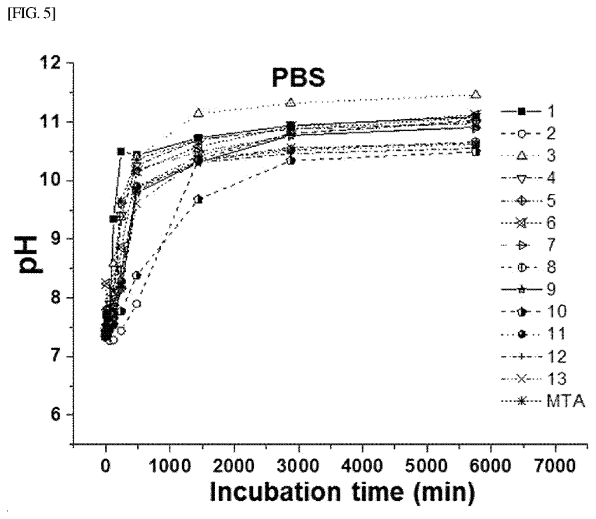

After mixing for 3 hours, the samples were placed in DW (pH 5.9) or PBS (pH 7.3), and the changes in pH were measured for up to 4 days. The changes in pH in the DW were similar in all cements, rapidly increased to pH 9 to pH 10 within 20 minutes, and a plateau of about pH 11 to pH 12 was reached after 8 hours (FIG. 4). Unlike the change in pH in DW, the change in pH of each sample measured in PBS was not significantly increased and maintained at a pH of about 7.5 to 8.5 until 2 hours, except in cement 1 (FIG. 5). After 2 days, all cements exceeded pH 10 in PBS, except for cement 10. On day 4, all cements including MTA, C3S, C2S, and C3A showed pH values of less than 11 except for cement 3 (C3S:C2S=60:40). It was confirmed that the pH value of the mixture of the MTA or the three powders (C3S, C2S, and C3A) had a range of 8 to 13 due to the release of hydroxyl ions during hydration of the cement according to a kind of powder and a liquid in which the powder and the extraction solution were mixed.

### EXPERIMENTAL EXAMPLE 3

### Measurement of Mechanical Properties

For a compressive strength test, a cylindrical sample with a diameter of 4 mm and a height of 6 mm was prepared by mixing for 2 minutes at an L/P ratio of 0.3. For a tensile strength test of a diameter, a disk sample with a diameter of 6 mm and a height of 4 mm was prepared by mixing for 2 minutes at an L/P ratio of 0.3. Thereafter, for 1, 7, 14, and 28 days at 37° C. and 100% relative humidity according to a previous protocol, the sample was treated in a simulated body fluid (SBF) at a constant temperature, and then compressive strength and tensile strength of a diameter were measured using a universal testing machine (Instron 3344; Instron Corp, Canton, Mass., USA) at a crosshead rate of 0.5 mm/min.

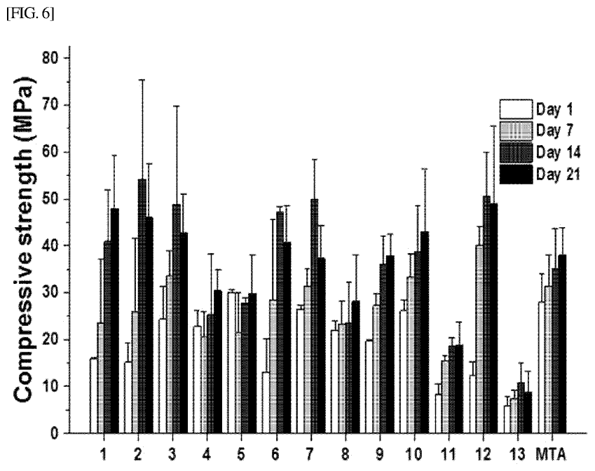

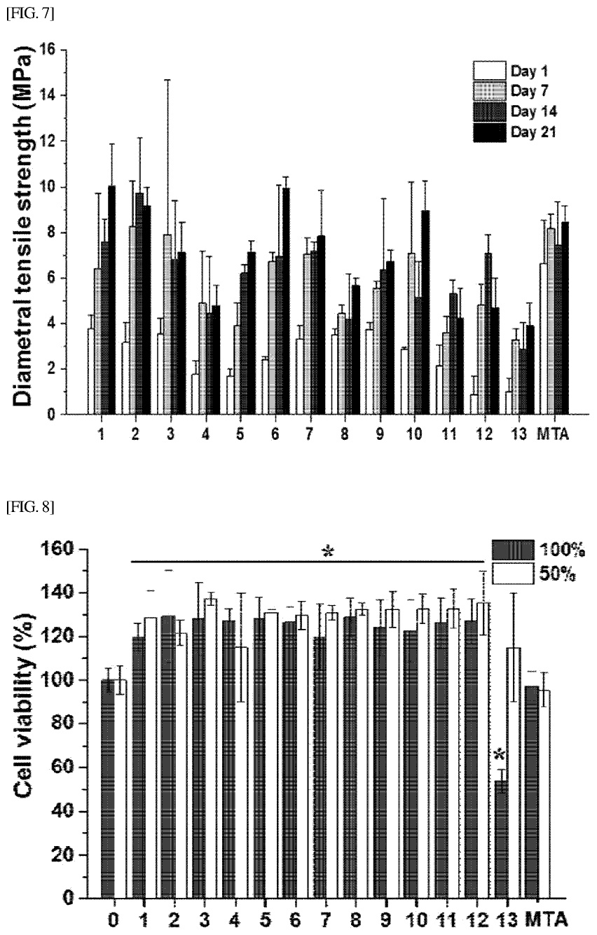

The mechanical properties were improved with increasing incubation time in all cements, and the maximum strength was increased by 2 to 4 times compared to the first day of culture (FIGS. 6 and 7).

### EXPERIMENTAL EXAMPLE 4

### Evaluation of Cell Compatibility

Rat mesenchymal stem cells (rMSCs) were collected from the femur and tibia bone marrow of 5-week-old male rats. The rMSCs were incubated in three subcultures. A total of 1000 cells were inoculated into a 96-well plate and incubated for 24 hours for cell attachment. Solutions (50% and 100%) eluted from each disk were placed 10 mL of a supplemented medium (DMEM containing 10% PBS and 1% Pen-Strep), applied to each well, and incubated at 37° C. for 3 days. Cells incubated in a culture medium without an eluent were used as a control. Cell proliferation was measured using a cell count kit-8 (CCK-8) according to the instructions of the manufacturer (Dojindo Molecular Technologies, Inc.). At the end of each culture time, 10 μL of a CCK-8 solution was added to each well of a 96-well plate, and the plate was incubated at 37° C. for 2 hours. The absorbance of each sample was measured at a wavelength of 450 nm using a microplate (iMark, BioRad). Each sample was repeatedly tested four times, and the results were normalized to a culture medium value of the control.

After 24 hours of co-culture, cell viability of 120% or more was observed in all cements, except cement 13 consisting of only C3A, as compared with a cell control, which was significantly increased (FIG. 8, P<0.05). Cement 13, consisting of only C3A, which contains aluminum, showed significantly mild cytotoxicity (about 60%, P<0.05), but cements containing 20% C3A (cement 8, cement 9, and cement 10) did not exhibit cytotoxicity but did increase cell viability.

### EXPERIMENTAL EXAMPLE 5

### Evaluation of Biocompatibility After Implanting In Vivo

All experimental procedures for animals were approved by the Institutional Animal Care and Use Committee (IACUC) of Dankook University (Approval DKU-13-031). A total of 36 11-week-old male Sprague-Dawley rats having a weight of 350 g to 400 g were used. 80 mg/kg of zoletil and 10 mg/kg of xylazine were injected intramuscularly into the right quadriceps femoris muscle of each rat. Lidocaine (0.5%) was injected locally into the premaxilla gingival of the upper jawbone. The rats were placed on their dorsal surface and disinfected with 10% povidone-iodine and 70% ethyl alcohol for aseptic surgery of surgical sites. All instruments were sterilized before surgery, and all four processes were performed aseptically according to a previous intentional injection method. The rats were kept in the room at 12° C. to 24° C. and 30% to 70% relative humidity at a cycle of 12 hours of day and 12 hours of night. The rats were fed a standard diet consisting of crushed pellet food and water. The rats were sacrificed at 4 weeks after surgery for sampling of surrounding tissues. Specimens were harvested from each animal at 4 weeks after surgery, and the samples were fixed in a 10% neutral buffered formaldehyde solution for at least 24 hours to 48 hours. Samples were reconstituted using an in vivo microcomputer tomography (μCT) system (Skyscan 1176, Skyscan, Aartselaar, Belgium) and NRecon μCT Skyscan reconstruction software to evaluate tissue recovery. Uncut cuttings and sections of the specimens were prepared in situ using an Exakt technique (Exakt Apparatebau, Norderstedt, Germany). A resin block specimen was cut into two halves along a long axis of cutting teeth. An initial cut section of 200 μm was ground to about 25 μm. The histology of hematoxylin and eosin (H & E) staining tissues was evaluated using an optical microscope (IX71, Olympus, Tokyo, Japan) and MetaMorph software (Molecular Devices, USA).

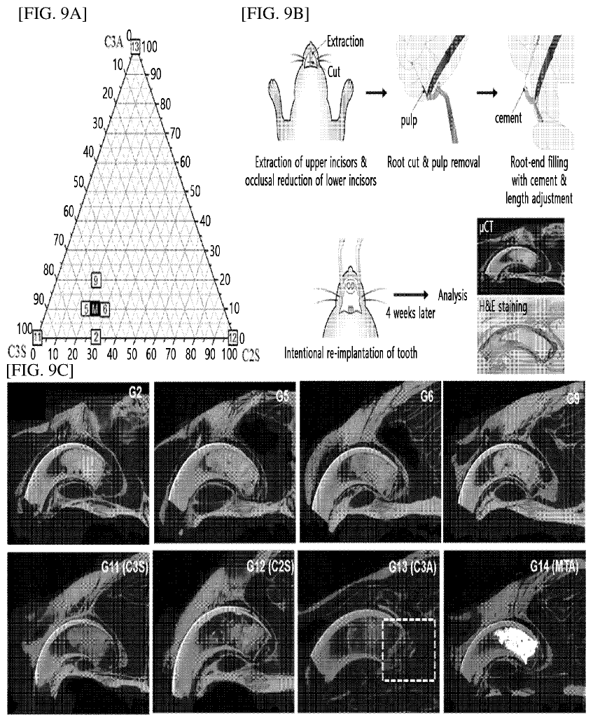

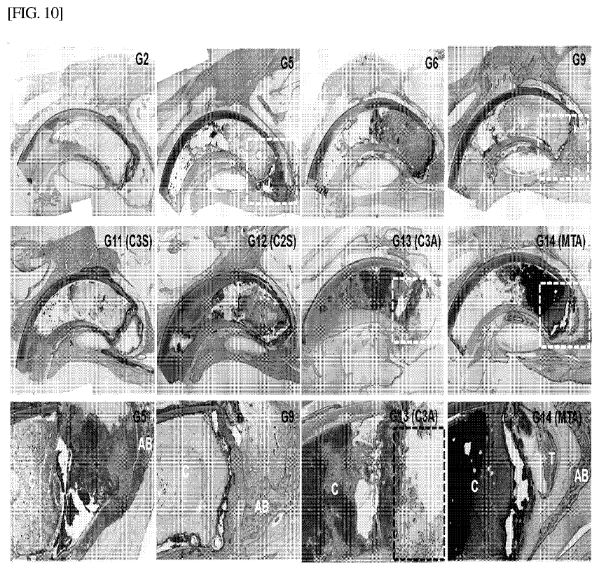

To closely examine the biocompatibility of the MTA, a single component of a cement and MTA composition (C2S, C3S, or C3A) with a composition similar to that of conventional MTA was used to intentionally implant the teeth after cementing root tips. Continuous processing of the extracted teeth, removal of the total pulp tissue of the root canal from an apical portion of the root canal, filling of each cement abdominally into an empty root canal, and re-injection of the tooth into the extracted region were performed using each of cement 2, cement 5, cement 6, cement 9, and cements consisting of C3S, C2S, C3A, and MTA (FIG. 9A).

This in vivo model mimics a clinical application of MTA to apical lesions of the tooth root, and the MTA directly contacts a tissue for tissue regeneration. According to a μCT diagram in FIG. 9C, it can be seen that in cement 13 consisting of only C3A, a red arrow indicates that thin radiopaque rays have been destroyed on a material-tissue interface, and a broken-line rectangle indicates the alveolar bone around the tooth, which has been damaged. In other words, not only the alveolar bone (broken-line rectangle) below the apex of the tooth but also a hard tissue layer of the proximal apex (white arrow) were destroyed, while other cements and the alveolar bone were separated by the same layer as a radiopaque bone. A thin hard tissue layer on an interface between the cement and the biotissue showed the bioactivity of the implanted material.

The MTA is a thin radiopaque layer preserved at the interface between the cement and the alveolar bone and exhibits significant radiopacity (more white).

Therefore, H & E staining was performed to observe a composition of the thin layer and an inflammatory response, and the tissue sections were observed. The MTA was located near the alveolar bone without significant inflammatory response (FIG. 10, MTT). In FIG. 10, a black line indicated by the dotted line indicates the destruction of the alveolar bone surrounding the tip of the tooth due to a severe inflammatory response. That is, in cement 13 consisting of only C3A, the inflammatory response of the apex was so severe that a thin hard tissue layer separating the material from the undamaged peri-apical lesion and the peripheral alveolar bone under the implanted material was destroyed (FIG. 10). In other cements, the same layer as thin osteo-dentin was observed on the interface, and the bioactivity of the cement was confirmed.

### Acknowledgement

This work has been supported by the Innopolis Foundation grant funded by the Korea government (MSIT) (No. 2017-DD-RD-0030-02).

Also, this work has been supported by Basic Science Research Program through the National Research Foundation of Korea (NRF) funded by the Ministry of Education (NRF-2019R1A6A1A11034536).

What is claimed is:

1. A dental cement composition comprising:
tricalcium silicate (C3S), dicalcium silicate (C2S), or both in which an individual particle has a mean diameter of 30 μm to 60 μm.

2. The dental cement composition of claim 1, further comprising:
tricalcium aluminate (C3A) of 25 wt % or less based on the total composition weight, where a particle has a mean diameter of 30 μm to 60 μm.

3. The dental cement composition of claim 1,
wherein the content of the tricalcium silicate is 45 wt % to 100 wt % based on the total composition weight.

4. The dental cement composition of claim 1,
wherein the content of the dicalcium silicate is 5 wt % to 100 wt % based on the total composition weight.

5. The dental cement composition of claim 1,
wherein the composition is used as a biomaterial for dental regeneration.

6. A method for preparation of an implant for regenerating a tissue comprising:
a step 1-1 of preparing a tricalcium silicate powder,
a step 1-2 of preparing a dicalcium silicate powder; and
a step 2 of adding an aqueous solvent to the powder prepared in the step 1-1, the powder prepared in the step 1-2, or a mixture thereof and hardening the powder.

7. The method of claim 6,
wherein based on the total powder, the content of the tricalcium silicate powder is 45 wt % to 85 wt %, and the content of the dicalcium silicate powder is 5 wt % to 45 wt %.

8. The method of claim 6, further comprising:
a step 1-3 of preparing a tricalcium aluminate powder before the step 2, wherein the step 2 is performed by additionally including the tricalcium aluminate powder in the mixture of the powders of the step 2.

9. The method of claim 8,
wherein the content of the tricalcium aluminate powder is 25 wt % or less based on the total powder.

10. The method of claim 6,
wherein a solvent/powder ratio (L/P) used in the step 2 is 0.2 to 0.4.

11. The method of claim 8,
wherein a solvent/powder ratio (L/P) used in the step 2 is 0.2 to 0.4.

12. The method of claim 6,
wherein the particles of the tricalcium silicate powder and the dicalcium silicate powder have an average diameter of 30 μm to 60 μm.

13. The method of claim 6,
wherein a hardening time of the implant is 10 minutes to 40 minutes.

14. The method of claim 8,
wherein a hardening time of the implant is 10 minutes to 40 minutes.

15. The method of claim 6,
wherein the implant is used for dental regeneration.

16. The method of claim 8,
wherein the implant is used for dental regeneration.

17. A kit for preparing a dental cement comprising:
a tricalcium silicate powder, a dicalcium silicate powder, or both.

18. The kit of claim 17, further comprising:
a tricalcium aluminate powder, an aqueous solvent, or both.

19. The kit of claim 18,
wherein the powders are provided individually or provided by a mixture of 45 wt % to 85 wt % of the tricalcium silicate powder, 5 wt % to 45 wt % of the dicalcium silicate powder, and 25 wt % or less of the tricalcium aluminate powder based on the total weight of the powders.
